(19) United States
(12) Patent Application Publication
(10) Pub. No.: US 2020/0323485 A1
(43) Pub. Date: Oct. 15, 2020

(54) SLEEP ANOMALY NOTIFICATION SYSTEM, SLEEP ANOMALY NOTIFICATION METHOD, AND PROGRAM

(71) Applicant: OPTIM CORPORATION, Saga (JP)

(72) Inventor: Shunji SUGAYA, Tokyo (JP)

(21) Appl. No.: 16/608,953

(22) PCT Filed: Apr. 28, 2017
(86) PCT No.: PCT/JP2017/016934

§ 371 Date: Oct. 28, 2019

(51) Int. Cl. A61B 5/00; G06K 9/62; G06K 9/00; G08B 21/06

(52) U.S. Cl. CPC A61B 5/4818; G06K 9/6256; G06K 9/00771; G06K 9/00248; G06K 9/00335; G08B 21/06; A61B 2576/00; A61B 5/0013; A61B 5/7267; A61B 5/7405; A61B 5/742; A61B 5/746; A61B 5/0077

(57) ABSTRACT

Provided is a sleep anomaly notification system. The system includes: an image acquisition module, which is configured to acquire sleep images of the person during sleep; an image analysis module, which is configured to perform an image analysis on the acquired sleep images; a sleep anomaly detection module, which is configured to detect whether a sleep anomaly happens to the person based on a result of the image analysis; an accept module, which is configured to accept registering of a mobile terminal of a caregiver of the person; a position acquisition module, which is configured to acquire a position of the registered mobile terminal; a determination module, which is configured to determine whether the acquired position is within a predefined range from a position of the person; and a notification module, which is configured to notify the sleep anomaly to the mobile terminal of the caregiver.

### TECHNICAL FIELD

The present invention relates to a sleep anomaly notification system, a sleep anomaly notification method, and a program, to acquire sleep images of a person during sleep, perform an image analysis, detect whether a sleep anomaly exists, and notify the sleep anomaly in condition that the sleep anomaly has been detected, so that the sleep anomaly can also be notified appropriately in condition that a caregiver to be notified is not around the person.

### BACKGROUND

A system for notifying a sleep anomaly is provided, which notifies a caregiver around with a sound or light when a person during sleep such as a patient in a hospital, an old person and an infant has a sleep anomaly such as sleeping in a prone posture (Patent document 1).

### LITERATURE IN THE RELATED ART

### Patent Document

Patent document 1: Japanese Patent Application No. JPH1199140 A.

### SUMMARY

### Problem to be Solved in the Present Invention

However, in the method of the patent document 1, there is a problem: even if the caregiver is notified by the sound and light, the caregiver cannot notice the notification if he is not around a notification unit.

In the present invention, in view of the above problem, the object of the present invention is to provide a sleep anomaly notification system, a sleep anomaly notification method, and a program to acquire sleep images of a person during sleep, perform image analysis, detect whether a sleep anomaly exists, and notify the sleep anomaly in condition that the sleep anomaly has been detected, so that the sleep anomaly can also be notified appropriately in condition that a caregiver to be notified is not around the person.

### Solution to the Problem

The present invention provides the following solutions.

The invention of a first characteristic provides a sleep anomaly notification system. The system includes: an image acquisition unit, which is configured to acquire sleep images of the person during sleep; an image analysis unit, which is configured to perform image analysis on the acquired sleep images; a sleep anomaly detection unit, which is configured to detect whether a sleep anomaly happens to the person based on a result of the image analysis; an accept unit, which is configured to accept registering of a mobile terminal of a caregiver of the person; a position acquisition unit, which is configured to acquire a position of the registered mobile terminal; a determination unit, which is configured to determine whether the acquired position is within a predefined range from a position of the person; and a notification unit, which is configured to: in response to determining that the sleep anomaly has been detected and the acquired position of the mobile terminal is not within the predefined range, notify the sleep anomaly to the mobile terminal of the caregiver.

According to the invention of the first characteristic, the sleep anomaly notification system includes: an image acquisition unit, which is configured to acquire sleep images of the person during sleep; an image analysis unit, which is configured to perform image analysis on the acquired sleep images; a sleep anomaly detection unit, which is configured to detect whether a sleep anomaly happens to the person based on a result of the image analysis; a accept unit, which is configured to accept registering of a mobile terminal of a caregiver of the person; a position acquisition unit, which is configured to acquire a position of the registered mobile terminal; a determination unit, which is configured to determine whether the acquired position is within a predefined range from a position of the person; and a notification unit, which is configured to: in response to determining that the sleep anomaly has been detected and that the acquired position of the mobile terminal is not within the predefined range, notify the sleep anomaly to the mobile terminal of the caregiver.

A type of the invention of the first characteristic is the sleep anomaly notification system, but even the sleep anomaly notification method and the program can also have the same effect and performance.

The invention of a second characteristic provides a sleep anomaly notification system, where in the sleep anomaly notification system as the invention of the first characteristic, the image acquisition unit acquires the sleep images from cameras disposed in a horizontal direction on both sides of a place where the person sleeps.

According to the invention of the second characteristic, in the sleep anomaly notification system as the invention of the first characteristic, the image acquisition unit acquires the sleep images from cameras disposed in a horizontal direction on both sides of a place where the person sleeps.

The invention of a third characteristic provides a sleep anomaly notification system, where in the sleep anomaly notification system as the invention of the first or second characteristic, the image acquisition unit performs the image analysis by performing machine learning using sleep images acquired previously as teacher data and customizing the sleep images acquired previously to be suitable for the person.

According to the invention of the third characteristic, in the sleep anomaly notification system as the invention of the first or second characteristic, the image acquisition unit performs the image analysis by performing machine learning using sleep images acquired previously as teacher data and customizing the sleep images acquired previously to be suitable for the person.

The invention of a fourth characteristic provides a sleep anomaly notification system, where in the sleep anomaly notification system as any one of inventions of the first to third characteristics, the sleep anomaly detection unit is configured to detect the sleep anomaly by performing machine learning using the result of the image analysis acquired previously as teacher data and customizing the result of the image analysis to be suitable for the person.

According to the invention of the fourth characteristic, in the sleep anomaly notification system as any one of inventions of the first to third characteristics, the sleep anomaly detection unit is configured to detect the sleep anomaly by performing machine learning using the result of the image analysis acquired previously as teacher data and customizing the result of the image analysis to be suitable for the person.

The invention of a fifth characteristic provides a sleep anomaly notification system, where in the sleep anomaly notification system as any one of inventions of the first to fourth characteristics, the sleep anomaly detection unit is configured to detect out the sleep anomaly by determining whether both nose and mouth of the person are blocked based on the result of the image analysis.

According to the invention of the fifth characteristic, in the sleep anomaly notification system as any one of inventions of the first to fourth characteristics, the sleep anomaly detection unit is configured to detect the sleep anomaly by detecting whether both nose and mouth of the person are blocked based on the result of the image analysis.

The invention of a sixth characteristic provides a sleep anomaly notification system, where in the sleep anomaly notification system as any one of inventions of the first to fifth characteristics, the sleep anomaly detection unit is configured to detect the sleep anomaly in condition that the result of the image analysis indicates that the person does not have any action within predefined time and thus no movement is observed.

According to the invention of the sixth characteristic, in the sleep anomaly notification system as any one of inventions of the first to fifth characteristics, the sleep anomaly detection unit is configured to detect out the sleep anomaly in condition that the result of the image analysis indicates that the person does not have any action within predefined time and thus no movement is observed.

The invention of a seventh characteristic provides a sleep anomaly notification system, where in the sleep anomaly notification system as any one of inventions of the first to sixth characteristics, the sleep anomaly detection unit is configured to detect out the sleep anomaly by using the case that the person does not have any action within the predefined time and thus no movement is observed as an anomaly and performing machine learning using the case as teacher data.

According to the invention of the seventh characteristic, in the sleep anomaly notification system as any one of inventions of the first to sixth characteristics, the sleep anomaly detection unit is configured to detect out the sleep anomaly by using the case that the person does not have any action within the predefined time and thus no movement is observed as an anomaly and performing machine learning using the case as teacher data.

The invention of an eighth characteristic provides a sleep anomaly notification system, where in the sleep anomaly notification system as any one of inventions of the first to seventh characteristics, the notification unit notifies the caregiver around the person with an alarming sound or alarming light in the case that the sleep anomaly has been detected and that the acquired position of the mobile terminal is within the predefined range.

According to the invention of the eighth characteristic, in the sleep anomaly notification system as any one of inventions of the first to seventh characteristics, the notification unit notifies the caregiver around the person with an alarming sound or alarming light in the case that the sleep anomaly has been detected and that the acquired position of the mobile terminal is within the predefined range.

The invention of a ninth characteristic provides a sleep anomaly notification system, where in the sleep anomaly notification system as any one of inventions of the first to eighth characteristics, the notification unit, in condition that the sleep anomaly has been detected and that the position of the mobile terminal is not acquired, notifies the caregiver around the person with the alarming sound or the alarming light or performs an action registered in advance in the sleep anomaly detection system.

According to the invention of the ninth characteristic, in the sleep anomaly notification system as any one of inventions of the first to eighth characteristics, the notification unit is configured to: in condition that the sleep anomaly has been detected and that the position of the mobile terminal is not acquired, notify the caregiver around the person with the alarming sound or the alarming light or performs an action registered in advance in the sleep anomaly detection system.

The invention of a tenth characteristic provides a sleep anomaly notification method. The sleep anomaly notification method includes the following steps: acquiring sleep images of the person during sleep; performing an image analysis on the acquired sleep images; detecting whether a sleep anomaly happens to the person based on a result of the image analysis; accepting registering of a mobile terminal of a caregiver of the person; acquiring a position of the registered mobile terminal; determining whether the acquired position is within a predefined range from a position of the person; and notifying the sleep anomaly to the mobile terminal of the caregiver in condition that the sleep anomaly has been detected and that the acquired position of the mobile terminal is not within the predefined range.

The invention of an eleventh characteristic provides a program. The program is configured to enable the sleep anomaly notification system to execute the following steps: acquiring sleep images of the person during sleep; performing an image analysis on the acquired sleep images; detecting whether a sleep anomaly happens to the person based on a result of the image analysis; accepting registering of a mobile terminal of a caregiver of the person; acquiring a position of the registered mobile terminal; determining whether the acquired position is within a predefined range from a position of the person; and notifying the sleep anomaly to the mobile terminal of the caregiver in condition that the sleep anomaly has been detected and that the acquired position of the mobile terminal is not within the predefined range.

### Effect of the Present Invention

According to the present invention, a sleep anomaly notification system, a sleep anomaly notification method, and a program can be provided to acquire sleep images of a person during sleep, perform image analysis, detect whether a sleep anomaly exists, and notify the sleep anomaly in condition that the sleep anomaly has been detected, so that the sleep anomaly can also be notified appropriately in condition that the caregiver to be notified is not around the person.

### BRIEF DESCRIPTION OF DRAWINGS

FIG. 1 is a schematic view of a preferred embodiment of the present invention.

FIG. 2 is a schematic view of function blocks of a camera 100, a computer 200 and a mobile terminal 300 and relationships thereof.

FIG. 3 is a flowchart of a case that the computer 200 performs image analysis on an image shot by the camera 100 and notifies the mobile terminal 300 of a sleep anomaly.

FIG. 4 is a schematic view of function blocks of a camera 100, a computer 200, a mobile terminal 300 and an alarming apparatus 500 and relationships thereof.

FIG. 5 is a flowchart of the computer 200 and the alarming apparatus 500 in condition that the sleep anomaly has been detected and that the mobile terminal 300 is within a predefined range (processing A).

FIG. 6 is a flowchart of the computer 200 and the alarming apparatus 500 in condition that the sleep anomaly has been detected and that position information of the mobile terminal 300 is not acquired (processing B).

FIG. 7 is a flowchart of a case that the computer 200 performs machine learning for image analysis of the sleep anomaly.

FIG. 8 is a schematic view of an example of a case that a caregiver 700 holding the mobile terminal 300 is not within the predefined range when the sleep anomaly has been detected.

FIG. 9 is a schematic view of an example of a case that the caregiver 700 holding the mobile terminal 300 is within the predefined range when the sleep anomaly has been detected.

FIG. 10 is a schematic view of an example of a case that position information of the mobile terminal 300 is not acquired when the sleep anomaly has been detected.

### DETAILED DESCRIPTION

Optimum embodiments for implementing the present invention will be described below with reference to the drawings. It is to be noted that the embodiments are merely examples and not intended to limit the scope of the present invention.

(Summary of the Sleep Anomaly Notification System)

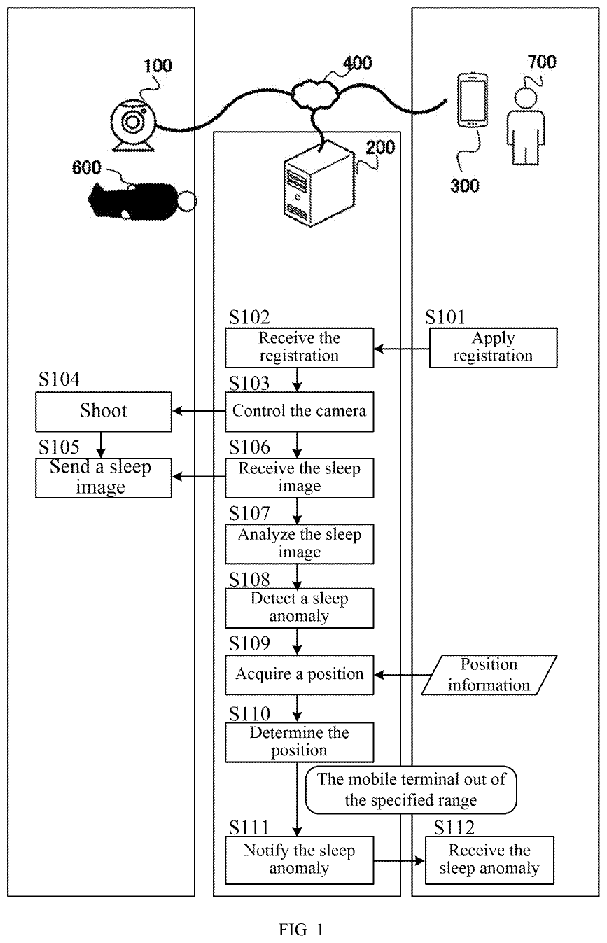

FIG. 1 is a schematic view of a preferred embodiment of the present invention. Base on FIG. 1, the summary of the present invention is described below. The sleep anomaly notification system includes a camera 100, a computer 200, a mobile terminal 300 and a communication network 400.

It is to be noted that in FIG. 1, the number of cameras 100 is not limited to one, but may be multiple. In addition, the computer 200 is limited to an actual apparatus, but may be a virtual apparatus.

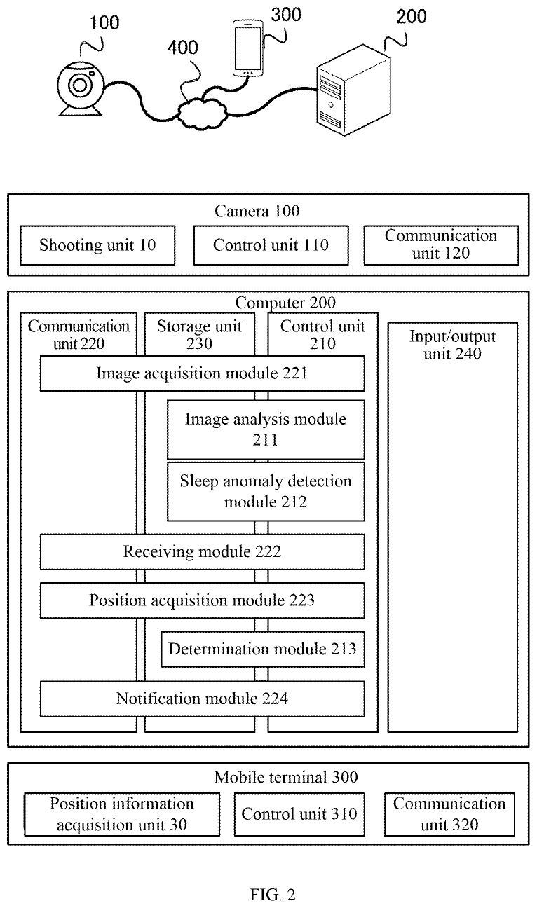

As shown in FIG. 2, the camera 100 includes a shooting unit 10, a control unit 110 and a communication unit 120. In addition, as shown in FIG. 2, the computer 200 includes a control unit 210, a communication unit 220, a storage unit 230 and an input/output unit 240. The control unit 210 cooperates with the storage unit 230 to implement the image analysis module 211, the sleep anomaly detection module 212 and the determination module 213. In addition, the communication unit 220 cooperates with the control unit 210 and the storage unit 230 to implement the image acquisition module 221, the receiving module 222, the position acquisition module 223 and the notification module 224. The mobile terminal 300 includes a position information acquisition unit 30, a control unit 310 and a communication unit 320. The communication network 400 may be a public communication network such as the Internet or may be a dedicated communication network, such that communications among the camera 100, the computer 200 and the mobile terminal 300 is implemented.

The camera 100 is a shooting apparatus capable of performing data communication with the computer 200 and is provided with shooting devices such as a shooting element, a lens and the like. The camera 100 is shown as a web camera as an example, but it may also be a shooting apparatus having necessary functions, such as a digital camera, a digital video camera, a camera mounted on an unmanned aerial vehicle, a camera mounted on a wearable device, a surveillance camera, an in-vehicle camera and a 360-degree camera.

The computer 200 is a computing apparatus capable of performing data communication with the camera. The computer 200 is shown as a desktop computer as an example, but besides a portable phone, a portable information terminal, a tablet terminal and a personal computer, the computer 200 may also be a laptop computer terminal, a slate terminal, an electronic book terminal, an electrical appliance such as a portable music player, and a wearable terminal such as smart glasses and a head-mounted display.

The mobile terminal 300 is a terminal apparatus held by a user using the sleep anomaly notification system. It is assumed that a caregiver 700 of a person 600 to be shot is the user using the sleep anomaly notification system. The mobile terminal 300 is shown as a smartphone as an example, but besides a portable phone, a portable information terminal, a tablet terminal and a personal computer, the mobile terminal 300 may also be a laptop computer terminal, a slate terminal, an electronic book terminal, an electrical appliance such as a portable music player, and a wearable terminal such as smart glasses and a head-mounted display.

In the sleep anomaly notification system shown in FIG. 1, the camera 100 is disposed in a horizontal direction of the person 600 on whom the sleep anomaly detection is performed, and can shoot the sleep images. Merely one camera 100 is shown, but ideally, two or more cameras 100 can be disposed in a manner in which the person 600 can be shot from his two sides. It is assumed that the person 600 is a person who needs to be took care, such as an infant, a patient in a hospital, an old person and the like. Particularly, in condition that the person 600 is assumed to be an infant, a prone posture of an infant with Sudden Infant Death Syndrome (SIDS) or the like needs to be detected to be a sleep anomaly, and notified to the caregiver 700. Therefore, shooting from both sides of the person 600 in the horizontal direction can further improve accuracy of image analysis for detecting the prone posture. In addition, the computer 200 is set to be a device which has completed the machine learning for image analysis of the sleep anomaly. The mobile terminal 300 is set to be a terminal held by the caregiver 700 of the person 600 to be shot.

First, a registration application to the sleep anomaly notification system is made from the mobile terminal 300 to the computer 200 (step S101). The above registration application is an application for setting the mobile terminal 300 held by the caregiver 700 of the person 600 to be a notification target of the sleep anomaly notification system. The application content for registration may include information of the camera 100, information of the person 600, and information of the caregiver 700.

Then, a receiving module 222 of the computer 200 receives the registration application from the mobile terminal 300 (step S102). The receiving module 222 sets the mobile terminal 300 held by the caregiver 700 of the person 600 who is shot by the camera 100 as the notification target of the sleep anomaly notification system. Correspondingly, in order to perform the sleep anomaly notification, position information of the mobile terminal 300 is set to be information that the computer 200 can acquire. In order to perform the setting, the computer 200 exchanges data with the mobile terminal 300 according to requirements.

Then, the computer 200 performs camera control on the camera 100 to shoot a dynamic or static sleep images of the person 600 (step S103). An instruction for starting to shoot the sleep images of the person 600 may directly come from the computer 200, and may also be an instruction received from the mobile terminal 300.

A shooting unit 10 of the camera 100 receives the control from the computer 200 to shoot the dynamic or static sleep images of the person 600 (step S104).

A control unit 110 of the camera 100 sends the shot sleep images to the computer 200 via a communication unit 120 (step S105).

An image acquisition module 221 of the computer 200 receives the sleep images from the camera 100 (step S106).

An image analysis module 211 of the computer 200 analyzes the sleep images from the camera 100 (step S107). The image analysis module 211 is set to be a module which has completed the machine learning for image analysis of the sleep anomaly. The method of machine learning for image analysis of the sleep anomaly will be described later.

A sleep anomaly detection module 212 of the computer 200 detects the sleep anomaly based on a result of the image analysis in the step S107 (step S108). The sleep anomaly, for example, may be a case that both nose and mouth of the person 600 are blocked, or a case that the person 600 does not have any action within the predefined time and that no movement is observed. In addition, the registration of the caregiver 700 may also be received to customize the sleep anomaly to be a case that figures of the infant are to enter his eyes, a case that the infant is to be fall off the bed, and the like.

In condition that the sleep anomaly has been detected, a position acquisition module 223 of the computer 200 acquires position information from the mobile terminal 300 (step S109).

In condition that the position information of the mobile terminal 300 can be acquired in the step S109, a determination module 213 of the computer 200 determines whether the mobile terminal 300 is within a predefined range (step S110).The predefined range is set to be within the periphery of the camera 100 and the person 600, or is set to be within the periphery of an alarming apparatus 500 which will be described later. In condition that the sleep anomaly is notified by the alarming apparatus 500, ideally, the predefined range is set to be a range where the notification can be normally recognized and notified.

In condition that the mobile terminal 300 is determined to be out of the predefined range in the step S110, a notification module 224 of the computer 200 notifies the mobile terminal 300 of the sleep anomaly (step S111).

Finally, the mobile terminal 300 receives the notification of the sleep anomaly via a communication unit 320, and prompts the caregiver 700 according to the setting (step S112). The method of prompting the sleep anomaly can be set in advance to be a sound, light, vibration, and display of dynamic or static images.

As described above, a sleep anomaly notification system, a sleep anomaly notification method, and a program can be provided according to the present invention, to acquire a sleep images of a person during sleep, perform image analysis, detect whether a sleep anomaly exists, and notify the sleep anomaly in condition that the sleep anomaly has been detected, so that even in condition that the caregiver to be notified is not around the person, the sleep anomaly can also be notified to the mobile terminal held by the caregiver, thereby appropriately notifying the sleep anomaly.

(Description of each Function)

FIG. 2 is a schematic view of function blocks of a camera 100, a computer 200 and a mobile terminal 300 and relationships thereof. The camera 100 includes a shooting unit 10, a control unit 110 and a communication unit 120. The computer 200 includes a control unit 210, a communication unit 220, a storage unit 230 and an input/output unit 240. The control unit 210 cooperates with the storage unit 230 to implement the image analysis module 211, the sleep anomaly detection module 212 and the determination module 213. In addition, the communication unit 220 cooperates with the control unit 210 and the storage unit 230 to implement the image acquisition module 221, the receiving module 222, the position acquisition module 223 and the notification module 224. The mobile terminal 300 includes a position information acquisition unit 30, a control unit 310 and a communication unit 320. The communication network 400 may be a public communication network such as the Internet or may be a dedicated communication network, such that communications among the camera 100, the computer 200 and the mobile terminal 300 is implemented.

The camera 100 is a shooting apparatus capable of performing data communication with the computer 200 and is provided with shooting devices such as a shooting element, a lens and the like. The camera 100 is shown as a web camera as an example, but it may also be a shooting apparatus having necessary functions, such as a digital camera, a digital video camera, a camera mounted on an unmanned aerial vehicle, a camera mounted on a wearable device, a surveillance camera, an in-vehicle camera and a 360-degree camera.

The camera 100, the shooting unit 10 is provided with shooting devices such as a lens, a shooting element, various buttons and a flashlight to shoot dynamic or static images. In addition, the shot image is a precise image having an amount of information necessary for the image analysis. In addition, the shooting unit 10 may also be set to control resolution, a camera angle and camera magnification during shooting.

The control unit 110 includes a central processing unit (CPU), a random access memory (RAM), a read only memory (ROM) and the like.

The communication unit 120 includes a device capable of communicating with another device, for example, a wireless fidelity (Wi-Fi) device based on IEEE802.11, a wireless device of the third generation (3G) mobile communication system or the fourth generation (4G) mobile communication system based on IMT-2000 standards and the like. The communication unit 120 may also be wired local area network (LAN) connection.

The computer 200 is a computing apparatus capable of performing data communication with the camera. The computer 200 is shown as a desktop computer as an example, but besides a portable phone, a portable information terminal, a tablet terminal and a personal computer, the computer 200 may also be a laptop computer terminal, a slate terminal, an electronic book terminal, an electrical appliance such as a portable music player, and a wearable terminal such as smart glasses and a head-mounted display.

The control unit 210 includes a CPU, a RAM, a ROM and the like. The control unit 210 cooperates with the storage unit 230 to implement the image analysis module 211, the sleep anomaly detection module 212 and the determination module 213.

The communication unit 220 includes a device capable of communicating with another device, for example, a Wi-Fi device based on IEEE802.11, a 3G or 4G wireless device based on IMT-2000 standards and the like. The communication unit 120 may also be wired LAN connection. The communication unit 220 cooperates with the control unit 210 and the storage unit 230 to implement the image acquisition module 221, the receiving module 222, the position acquisition module 223 and the notification module 224.

The storage unit 230 includes a storage unit for data implemented by a hard disk or a semiconductor memory, and stores data required for processing shot images, teacher data, image analysis results and the like. In addition, the storage unit 230 may also include a database of teacher data of sleep images.

The input/output unit 240 is set to have functions required in condition that an operator operates the sleep anomaly notification system via the computer 200. The input/output unit 240 for implementing input, for example, may include: a liquid crystal display implementing a touch function, a keyboard, a mouse, a digital panel, hardware buttons on the apparatus, a microphone for sound recognition and the like. In addition, the input/output unit 240 for implementing output, for example, may adopt the manner for outputting display and sound via a liquid display, a display of a PC, and projection of a projector. The function of the present invention is not specially limited by the input/output mode.

The mobile terminal 300 is a terminal apparatus held by a user using the sleep anomaly notification system. The mobile terminal 300 is shown as a smartphone as an example, but besides a portable phone, a portable information terminal, a tablet terminal and a personal computer, the mobile terminal 300 may also be a laptop computer terminal, a slate terminal, an electronic book terminal, an electrical appliance such as a portable music player, and a wearable terminal such as smart glasses and a head-mounted display.

The mobile terminal 300 includes a position information acquisition unit 30 which can acquire information of latitude, longitude and height of the mobile terminal 300 via the function of Global Positioning System (GPS). The method for acquiring position information is limited to the GPS, but the position information may also be acquired in a wireless communication manner such as Wi-Fi, Bluetooth, near-field communication (NFC), 3G, 4G, and Long Term Evolution (LTE). In addition, acquisition of position information corresponding to each communication manner is not limited in the present patent, and can use existing techniques.

The control unit 310 includes a CPU, a RAM, a ROM and the like.

The communication unit 320 includes a device capable of communicating with another device, for example, a Wi-Fi device based on IEEE802.11, a 3G or 4G wireless device based on IMT-2000 specification and the like.

(Sleep Anomaly Notification Processing)

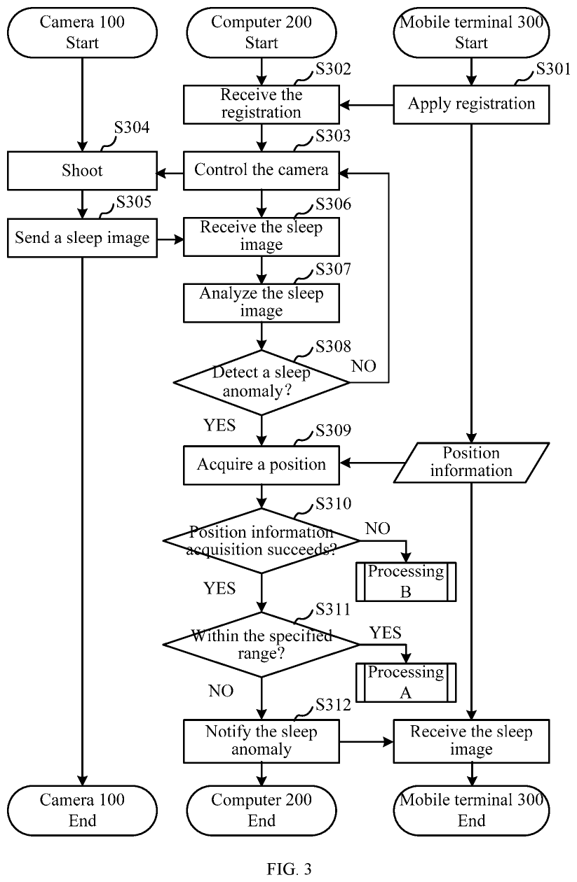

FIG. 3 is a flowchart of a case that the computer 200 performs image analysis on an image shot by the camera 100 and notifies the mobile terminal 300 of a sleep anomaly. Processing performed by each of the above modules will be described in conjunction with the processing.

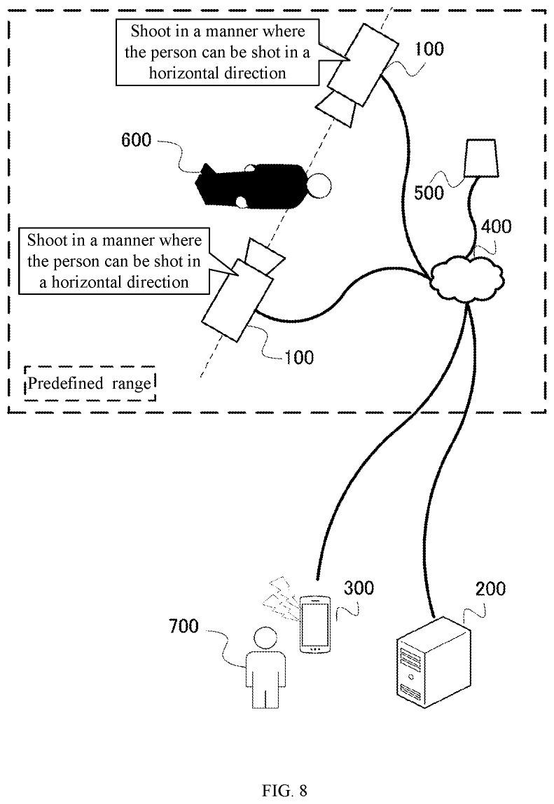

FIG. 8 is a schematic view of an example of a case that a caregiver 700 holding the mobile terminal 300 is not within the predefined range when the sleep anomaly notification system has detected the sleep anomaly. The camera 100 is disposed in a horizontal direction of the person 600 on whom the sleep anomaly detection is performed, and can shoot the sleep images. Merely one camera 100 is shown, but ideally, two or more cameras 100 can be disposed in a manner in which the person 600 can be shot from his two sides. It is assumed that the person 600 is a person who needs to be took care, such as an infant, a patient in a hospital, an old person and the like. Particularly, in condition that the person 600 is assumed to be an infant, a prone posture of an infant with SIDS or the like needs to be detected to be a sleep anomaly, and notified to the caregiver 700. Therefore, shooting from both sides of the person 600 in the horizontal direction can further improve precision of image analysis for detecting the prone posture. In addition, the computer 200 is set to be a device which has completed the machine learning for image analysis of the sleep anomaly. The mobile terminal 300 is set to be a terminal held by the caregiver 700 of the person 600 to be shot. The alarming apparatus 500 shown in FIG. 8 will be described later. The communication network 400 may be a public communication network such as the Internet or may be a dedicated communication network, such that communications among the camera 100, the computer 200, the mobile terminal 300 and the alarming apparatus 500 is implemented.

Back to the flowchart in FIG. 3, and first, a registration application to the sleep anomaly notification system is made from the mobile terminal 300 to the computer 200 (step S301). The above registration application is an application for setting the mobile terminal 300 held by the caregiver 700 of the person 600 to be a notification target of the sleep anomaly notification system. The application content for registration may also include information of the camera 100, information of the person 600, and information of the caregiver 700.

Then, a receiving module 222 of the computer 200 receives the registration application from the mobile terminal 300 (step S302). The receiving module 222 sets the mobile terminal 300 held by the caregiver 700 of the person 600 who is shot by the camera 100 as the notification target of the sleep anomaly notification system. Correspondingly, in order to perform the sleep anomaly notification, position information of the mobile terminal 300 is set to be information that the computer 200 can acquire. In order to perform the setting, the computer 200 exchanges data with the mobile terminal 300 according to requirements.

Then, the computer 200 performs camera control on the camera 100 to shoot a dynamic or static sleep images of the person 600 (step S303). An instruction for starting to shoot the sleep images of the person 600 may directly come from the computer 200, and may also be an instruction received from the mobile terminal 300.

A shooting unit 10 of the camera 100 is configured to receive the control from the computer 200 to shoot the dynamic or static sleep images of the person 600 (step S304).

A control unit 110 of the camera 100 is configured to send the shot sleep images to the computer 200 via a communication unit 120 (step S305).

An image acquisition module 221 of the computer 200 is configured to receive the sleep images from the camera 100 (step S306).

An image analysis module 211 of the computer 200 is configured to analyze the sleep images from the camera 100 (step S307). The image analysis module 211 is set to be a module which has completed the machine learning for image analysis of the sleep anomaly. The method of machine learning for image analysis of the sleep anomaly will be described later.

A sleep anomaly detection module 212 of the computer 200 is configured detect the sleep anomaly based on a result of the image analysis in the step S107 (step S308). The sleep anomaly, for example, may be a case that both nose and mouth of the person 600 are blocked, or a case that the person 600 does not have any action within the predefined time and that no movement is observed. In addition, the registration of the caregiver 700 may also be received to customize the sleep anomaly to be a case that figures of the infant are to enter his eyes, a case that the infant is to be fall off the bed, and the like.

In condition that the sleep anomaly has been detected, a position acquisition module 223 of the computer 200 is configured to acquire position information from the mobile terminal 300 (step S309). In condition that no sleep anomaly has been detected, return to the step S303 to continue to acquire the sleep images.

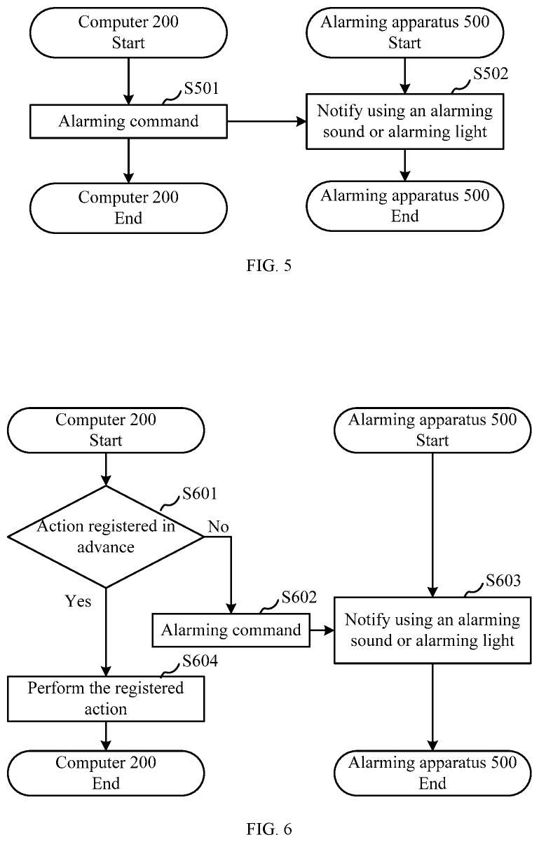

The position acquisition module 223 of the computer 200 is configured to determine whether the acquisition of the position information from the mobile terminal 300 succeeds (step S310). In condition that the acquisition of the position information succeeds, go to step S311, and in condition that the acquisition of the position information fails, go to processing B. The processing B will be described later as the description about FIG. 6.

In condition that the acquisition of the position information succeeds, a determination module 213 of the computer 200 determines whether the mobile terminal 300 is within a predefined range (step S311). The predefined range is set to be within the periphery of the camera 100 and the person 600, or is set to be within the periphery of an alarming apparatus 500 which will be described later. In condition that the sleep anomaly is notified by the alarming apparatus 500, ideally, the predefined range is set to be a range where the notification can be normally recognized and notified. In condition that the mobile terminal 300 is determined to be within the predefined range, go to processing A, and in condition that the mobile terminal 300 is determined to be out of the predefined range, go to step S312. The processing A will be described later as the description about FIG. 5.

In step S311, in condition that the mobile terminal 300 is determined to be out of the predefined range, a notification module 224 of the computer 200 notifies the mobile terminal 300 of the sleep anomaly (step S312).

Finally, the mobile terminal 300 receives the notification of the sleep anomaly via a communication unit 320, and prompts the caregiver 700 according to the setting (step S313). The method of prompting the sleep anomaly can be set in advance to be a sound, light, vibration, and display of dynamic or static images.

Although not shown in the flowchart in FIG. 3, an instruction for ending the shooting of the sleep images of the person 600 may directly come from the computer 200, and may also be an instruction received from the mobile terminal 300. In condition that the computer has received the instruction for ending the shooting, end the cyclic processing from step S303 to S308, and end the sleep anomaly notification system.

As described above, a sleep anomaly notification system, a sleep anomaly notification method, and a program can be provided according to the present invention to acquire a sleep images of a person during sleep, perform image analysis, detect whether a sleep anomaly exists, and notify the sleep anomaly in condition that the sleep anomaly has been detected, so that even in condition that the caregiver to be notified is not around the person, the sleep anomaly can also be notified to the mobile terminal held by the caregiver, thereby appropriately notifying the sleep anomaly.

(Sleep Anomaly Notification Processing via the Alarming Apparatus)

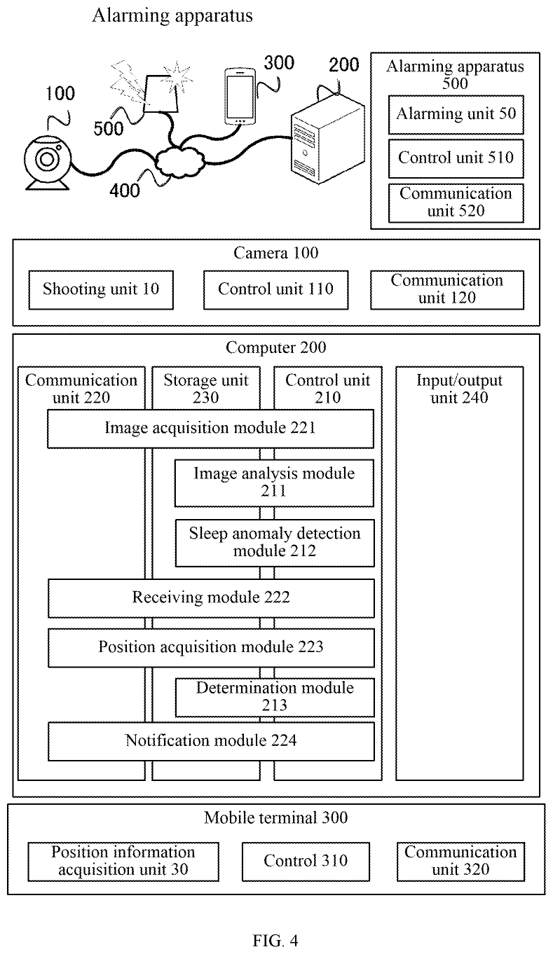

FIG. 4 is a schematic view of function blocks of a camera 100, a computer 200, a mobile terminal 300 and an alarming apparatus 500 and relationships thereof. Besides the composition in FIG. 2, the system further includes an alarming apparatus 500. The alarming apparatus 500 includes an alarming unit 50, a control unit 510 and a communication unit 520. The communication network 400 may be a public communication network such as the Internet or may be a dedicated communication network, such that communications among the camera 100, the computer 200, the mobile terminal 300 and the alarming apparatus 500 is implemented.

In the alarming apparatus 500, the alarming unit 50 has composition which can notify the caregiver 700 of the sleep anomaly using an alarming sound or alarming light.

The control unit 510 includes a CPU, a RAM, a ROM and the like, and receives instructions from the computer to enable the alarming unit 50 to work.

The communication unit 520 includes a device capable of communicating with another device, for example, a Wi-Fi device based on IEEE802.11, a 3G or 4G wireless device based on IMT-2000 specification and the like. The communication unit 520 may also be wired LAN connection. The alarming unit 50 receives an operation instruction from the computer 200 via the communication unit 520.

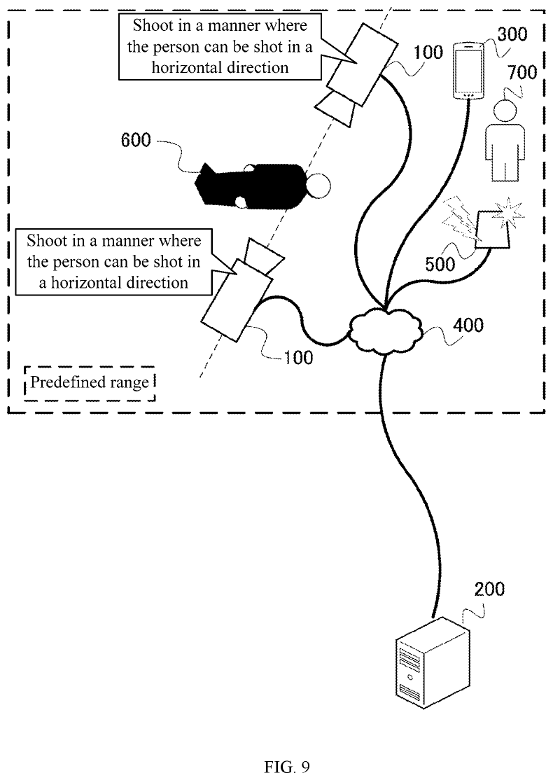

FIG. 9 is a schematic view of an example of a case that the caregiver 700 holding the mobile terminal 300 is within the predefined range when the sleep anomaly has been detected. The camera 100 is disposed in a horizontal direction of the person 600 on whom the sleep anomaly detection is performed in a case the sleep anomaly exists, and can shoot the sleep images. Ideally, as shown in the figure, two or more cameras 100 can be disposed in a manner in which the person 600 can be shot from his two sides. In addition, the computer 200 is set to be a device which has completed the machine learning for image analysis of the sleep anomaly. The mobile terminal 300 is set to be a terminal held by the caregiver 700 of the person 600 to be shot. The alarming apparatus 500 uses the alarming sound or the alarming light to notify the caregiver 700 around of the sleep anomaly. The communication network 400 may be a public communication network such as the Internet or may be a dedicated communication network, such that communications among the camera 100, the computer 200, the mobile terminal 300 and the alarming apparatus 500 is implemented.

FIG. 5 is a flowchart of the computer 200 and the alarming apparatus 500 in condition that the sleep anomaly has been detected and that the mobile terminal 300 is within a predefined range, which is equal to a case of performing processing A in the flowchart of FIG. 3 under a situation shown in FIG. 9.

In condition that the sleep anomaly has been detected and that the mobile terminal 300 is determined to be within the predefined range, that is, in condition that the caregiver 700 is considered to be within the range where he can normally recognize the sleep anomaly notification of the alarming apparatus 500, the notification module 224 of the computer 200 notifies the alarming apparatus 500 of an alarming command (step S501).

The alarming apparatus 500 receives the alarming command via the communication unit 520, enables the alarming unit 50 to work according to an instruction from the control unit 510, and notifies the sleep anomaly using the alarming sound or the alarming light (step S502). The alarming apparatus 500 may also notify the sleep anomaly using both the alarming sound and the alarming light. In addition, the function of the alarming apparatus 500 may also be displayed via vibration, words, etc.

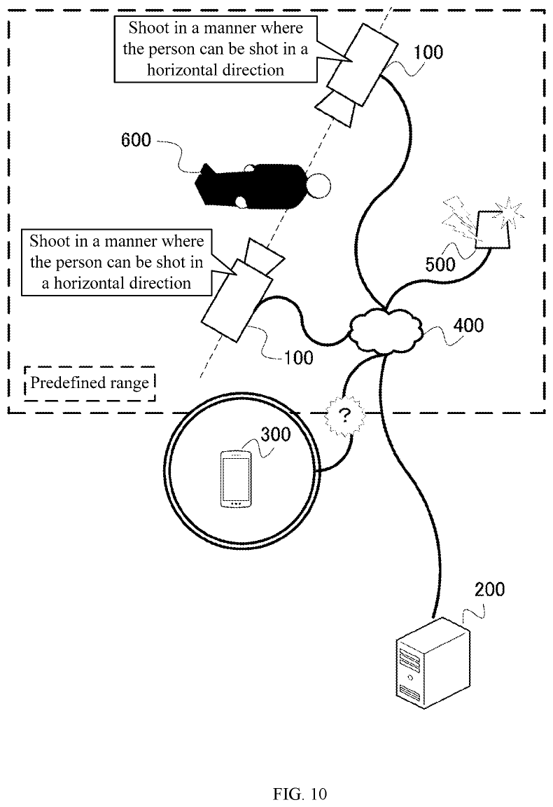

In addition, FIG. 10 is a schematic view of an example of a case that position information of the mobile terminal 300 is not acquired when the sleep anomaly has been detected. The camera 100 is disposed in a horizontal direction of the person 600 on whom the sleep anomaly detection is performed in a case the sleep anomaly exists, and can shoot the sleep images. Ideally, as shown in the figure, two or more cameras 100 can be disposed in a manner in which the person 600 can be shot from his two sides. In addition, the computer 200 is set to be a device which has completed the machine learning for image analysis of the sleep anomaly. The mobile terminal 300 is set to be a terminal held by the caregiver 700 of the person 600 to be shot. However, in condition that the position information of the mobile terminal 300 cannot be acquired due to exhaustion and communication failure, the alarming apparatus 500 uses the alarming sound or the alarming light to notify the caregiver 700 around of the sleep anomaly. The communication network 400 may be a public communication network such as the Internet or may be a dedicated communication network, such that communications among the camera 100, the computer 200, the mobile terminal 300 and the alarming apparatus 500 is implemented.

FIG. 6 is a flowchart of the computer 200 and the alarming apparatus 500 in condition that the sleep anomaly has been detected and that position information of the mobile terminal 300 is not acquired, which is equal to a case of performing processing B in the flowchart of FIG. 3 under a situation shown in FIG. 10.

In condition that the sleep anomaly has been detected and that the position information of the mobile terminal 300 cannot be acquired, that is, in condition that it is unclear whether the caregiver 700 is within the range where he can normally recognize the sleep anomaly notification of the alarming apparatus 500, the notification module 224 of the computer 200 confirms whether an action for failure of acquiring the position information of the mobile terminal is registered in advance (step S601).

In condition that the action for the failure of acquiring the position information of the mobile terminal 300 is registered, go to step S604, while in a case of no registered action, the notification module 224 notifies the alarming apparatus of the alarming command (step S602).

In condition that the computer 200 notifies the alarming command, the alarming apparatus 500 receives the alarming command via the communication unit 520, enables the alarming unit 50 to work according to an instruction from the control unit 510, and notifies the sleep anomaly using the alarming sound or the alarming light (step S603). The alarming apparatus 500 may also notify the sleep anomaly using both the alarming sound and the alarming light. In addition, the function of the alarming apparatus 500 may also be displayed via vibration, words, etc.

In condition that the action for the failure of acquiring the position information of the mobile terminal 300 is registered, the notification module 224 performs the registered action (step S604). The registered action, for example, may be alarming command notification to the alarming apparatus 500, notification to another mobile terminal, notification to a manager of the sleep anomaly notification system, notification to a security company, or notification to a hospital or a medical facility, or may be more than one of the above registered actions.

As described above, a sleep anomaly notification system, a sleep anomaly notification method, and a program can be provided according to the present invention to acquire a sleep images of a person during sleep, perform image analysis, detect whether a sleep anomaly exists, and notify the sleep anomaly in condition that the sleep anomaly has been detected. Therefore, in condition that the caregiver to be notified is not around the person, the sleep anomaly can be notified to the mobile terminal held by the caregiver, in condition that the caregiver to be notified is around the person, the sleep anomaly can be notified by the alarming apparatus; and in condition that it is unclear whether the caregiver to be notified is around the person, the sleep anomaly can be notified through the alarming of the alarming apparatus 500 or through the action registered in advance.

(Machine Learning Processing of Image Analysis of the Sleep Anomaly)

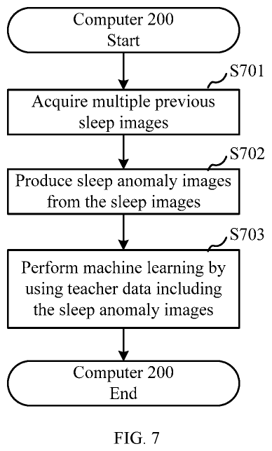

FIG. 7 is a flowchart of a case that the computer 200 performs machine learning for image analysis of the sleep anomaly.

The control unit 210 of the computer 200 acquires multiple sleep images acquired previously from the storage unit 230 (step S701). The acquired sleep images can further improve accuracy of image analysis by using an image of the person 600 on whom the sleep analysis detection is performed.

In condition that the acquired sleep images include sufficient sleep anomaly images, the acquired sleep images are used as teacher data images during sleep anomaly, but it is usually considered that the sleep images acquired previously do not include such sufficient sleep anomaly images. Therefore, the control unit 210 produces sleep anomaly images based on the acquired sleep images (step S702). The produced sleep anomaly image, for example, may be a case that both nose and mouth of the person 600 are blocked, or a case that the person 600 does not have any action within the predefined time and that no movement is observed. Particularly, since the risk of SIDS is associated with an apnea attack lasting for more than a few seconds to about 20 seconds, it is possible to effectively detect a sleep anomaly by producing a sleep anomaly image matching the age of the person 600, in which the sleep anomaly is detected in predefined time.

Finally, the control unit 210 performs machine learning by using teacher data including the sleep anomaly images produced in the sleep images acquired previously (step S703).

As described above, a sleep anomaly notification system, a sleep anomaly notification method, and a program can be provided according to the present invention, so as to, in a case of performing machine learning for image analysis of the sleep anomaly, produce sleep anomaly images based on sleep images acquired previously and perform machine learning by using teacher data including a sufficient number of sleep anomaly images, thereby effectively adding the teacher data and further improving detection accuracy of the sleep anomaly of image analysis.

The above units and functions are implemented by reading and executing specified programs by a computer (including a CPU, an information processing apparatus and various terminals). The programs can be provided, for example, in the following manner: being provided from a computer via a network (such as software as a service (SaaS)), or being provided by being recorded on a computer-readable recording medium such as a floppy disk, a compact disk (CD) (a CD-ROM, etc.), a digital versatile disc (DVD) (a DVD-ROM, a DVD-RAM, etc.), a compact memory, or the like. In this case, the computer reads the programs from the recording medium and transfers the programs to an internal storage device or an external storage device for storage and execution. In addition, for example, the programs may also be recorded in advance on a storage device (recording medium) such as a magnetic disk, an optical disk or a magneto-optical disk and provided for the computer via a communication line.

The embodiments of the present invention have been described above, but the present invention is not limited to the above embodiments. In addition, the effects described in the embodiments of the present invention are merely illustrative of the best effects produced by the present invention, and the effects of the present invention are not limited to the effects described in the embodiments of the present invention.

### LIST OF REFERENCE NUMBERS

100 camera, 200 computer, 300 mobile terminal, 400 communication network, 500 alarming apparatus, 600 person, and 700 caregiver.

1. A sleep anomaly notification system, comprising:
an image acquisition unit, which is configured to acquire sleep images of a person during sleep;
an image analysis unit, which is configured to perform an image analysis on the acquired sleep images;
a sleep anomaly detection unit, which is configured to detect whether a sleep anomaly happens to the person based on a result of the image analysis;
an accept unit, which is configured to accept registering of a mobile terminal of a caregiver of the person;
a position acquisition unit, which is configured to acquire a position of the registered mobile terminal;
a determination unit, which is configured to determine whether the acquired position is within a predefined range from a position of the person; and
a notification unit, which is configured to: in response to determining that the sleep anomaly has been detected and the acquired position of the mobile terminal is not within the predefined range, notify the sleep anomaly to the mobile terminal of the caregiver.

2. The sleep anomaly notification system of claim 1, wherein
the image acquisition unit is configured to acquire the sleep images from cameras disposed in a horizontal direction on both sides of a place where the person sleeps.

3. The sleep anomaly notification system of claim 1, wherein
the image analysis unit is configured to perform the image analysis by performing machine learning using sleep images acquired previously as teacher data and customizing the sleep images acquired previously to be suitable for the person.

4. The sleep anomaly notification system of claim 1, wherein
the sleep anomaly detection unit is configured to detect the sleep anomaly by performing machine learning using the result of the image analysis as teacher data and customizing the result of the image analysis to be suitable for the person.

5. The sleep anomaly notification system of claim 1, wherein
the sleep anomaly detection unit is configured to detect the sleep anomaly by determining whether both nose and mouth of the person are blocked based on the result of the image analysis.

6. The sleep anomaly notification system of claim 1, wherein
the sleep anomaly detection unit is configured to detect out the sleep anomaly in condition that the result of the image analysis indicates that the person does not have any action within predefined time and thus no movement is observed.

7. The sleep anomaly notification system of claim 1, wherein
the sleep anomaly detection unit is configured to detect out the sleep anomaly by using a case that the person does not have any action within the predefined time and thus no movement is observed as an anomaly and performing machine learning by using the case as teacher data.

8. The sleep anomaly notification system of claim 1, wherein
the notification unit is configured to: in response to determining that the sleep anomaly has been detected and the acquired position of the mobile terminal is within the predefined range, notify the caregiver around the person with an alarming sound or alarming light.

9. The sleep anomaly notification system of claim 1, wherein
the notification unit is configured to: in response to determining that the sleep anomaly has been detected and that the position of the mobile terminal is not acquired, notify the caregiver around the person with the alarming sound or the alarming light, or performs an action registered in advance in the sleep anomaly detection system.

10. An anomaly notification method, comprising following steps:
acquiring sleep images of a person during sleep;
performing an image analysis on the acquired sleep images;
detecting whether a sleep anomaly happens to the person based on a result of the image analysis;
accepting registering of a mobile terminal of a caregiver of the person;
acquiring a position of the registered mobile terminal;
determining whether the acquired position is within a predefined range from a position of the person; and
notifying the sleep anomaly to the mobile terminal of the caregiver in response to determining that the sleep anomaly has been detected and that the acquired position of the mobile terminal is not within the predefined range.

11. A computer-readable program, which is configured to enable a sleep anomaly notification system to execute following steps:
acquiring sleep images of a person during sleep;
performing image analysis on the acquired sleep images;
detecting whether a sleep anomaly happens to the person based on a result of the image analysis;
accepting registering of a mobile terminal of a caregiver of the person;
acquiring a position of the registered mobile terminal;
determining whether the acquired position is within a predefined range from a position of the person; and
notifying the sleep anomaly to the mobile terminal of the caregiver in a response to determining that the sleep anomaly has been detected and that the acquired position of the mobile terminal is not within the predefined range.
